(19) United States
(12) Patent Application Publication
(10) Pub. No.: US 2022/0143216 A1
(43) Pub. Date: May 12, 2022

(54) RECOMBINANT VECTORS COMPRISING ARYLSULFATASE A AND THEIR USES IN STEM CELL THERAPY FOR THE TREATMENT OF METACHROMATIC LEUKODYSTROPHY

(71) Applicant: VERSITECH LIMITED, Hong Kong (CN)

(72) Inventor: Qizhou LIAN, Hong Kong (CN)

(21) Appl. No.: 17/434,513

(22) PCT Filed: Feb. 28, 2020
(86) PCT No.: PCT/CN2020/077088

§ 371 Date: Aug. 27, 2021

Related U.S. Application Data
(60) Provisional application No. 62/813,411, filed on Mar. 4, 2019.

(51) Int. Cl. A61K 48/00; C12N 15/86; A61P 25/28; A61P 3/00

(52) U.S. Cl. CPC A61K 48/005; C12N 15/86; A61P 25/28; C12N 2830/48; C12N 2740/16043; C12N 2830/38; C12N 2830/40; A61P 3/00

(57) ABSTRACT

Provided are recombinant lentiviral vectors comprising an expression cassette comprising a nucleic acid construct expressing an ARSA gene. The vectors are useful in gene therapy for the treatment of Metachromatic Leukodystrophy. Provided are methods of producing the vectors. Provided are multipotent stem cells comprising the vectors. Also provided are methods of culturing the stem cells to maintain their multipotency.

### 1. INTRODUCTION

Described herein are recombinant lentiviral vectors comprising an expression cassette comprising a nucleic acid construct expressing an arylsulfatase A gene. The vectors are useful in gene therapy for the treatment of Metachromatic Leukodystrophy. Also disclosed are methods of producing the vectors. Provided herein are multipotent stem cells comprising the vectors. Also provided are methods of culturing the stem cells to maintain their multipotency. media and culture method of maintaining the multipotency of the transfected stem cells.

### 2. BACKGROUND

Metachromatic leukodystrophy (MILD) is a rare lysosomal storage disorder which is caused by mutations in arylsulfatase A (ARSA) gene that leads to deficiency of ARSA. This enzyme catalyzes the first step in the degradation pathway sulfatide to galacto-cerebroside. Deficiency of ARSA causes sulfatide accumulation in oligodendrocytes, microglia, and certain neurons of the central nervous system (CNS), and in Schwann cells and macrophages of the peripheral nervous system (PNS). This results in microglia activation, progressive degeneration, demyelination, and various lethal neurological symptoms such as severe progressive motor and cognitive impairment.

Three types of MLD are observed: 1. Infant onset MLD in early to late infancy (up to age 2), which is most commonly observed for 50-60% of all occurrence; 2. Juvenile onset MLD in early to late adolescence (age 2 to 16), 30-40% of all occurrence; and 3. Adult onset in adulthood after age 16, 5-10% of all occurrence. It is estimated that 1.4-1.8 babies out of 100,000 births carry MLD, which is approximately 1,900 new patients every year. Currently, there are around 41,000 patients in the world. There is no typical symptom in early stages. Over 90% of patients get incorrect diagnosis and miss the opportunity to treat the disease in a timely manner. If left untreated, patients can suffer paralysis and blindness within 1-2 years in late infancy or 6-8 years in juvenile.

There is currently no clinically proven cure for MLD, and treatment for most patients with MLD was confined to supportive care until recently. Several treatment options are now being developed and are at clinical trial stage. Most are enzyme replacement therapy (ERT) and hematopoietic stem cell transplantation (HSCT). Enzyme replacement therapy (ERT) with a supply of functional ARSA has been challenging because ARSA is a high molecular weight protein that is unable to penetrate the blood-brain barrier (BBB), thereby showing limited effect. If hematopoietic stem cells cannot cross the BBB, they cannot differentiate to microglial cells which secrete limited ARSA enzyme that is taken up by the recipient neural cells.

Hematopoietic stem cell transplantation (HSCT) can benefit selected subsets of patients with lysosomal storage diseases. Also, stem cell transplantation removed the need for the patient to go through chemotherapy, termed Busulfan-based conditioning, to remove original bone marrow cells and generate space for gene corrected-stem cell growth. This procedure potentially introduced the risk of multiple lesions including the neural system. By careful titration of therapy, the conditioning treatment can be more tolerable and the patient can make a rapid recovery following transplantation without obvious side-effects. Also, transplantation of allogeneic cells carries the risk of graft-versus host disease (GvHD), which can be a cause of extensive morbidity. HSCT using umbilical cord blood (UCB) from matched unrelated donors holds reduced risk of acute or chronic GvHD compared with using bone marrow (BM).

However, there is a higher probability of engraftment failure using UCB as a result of its lower cell dose and immunologic immaturity (Kamani et al. (2012) Biol. Blood Marrow Transplant. 18(8): 1265-1272; Locatelli and Pagliara (2012) Pediatr. Blood Cancer. 59(2): 372-376). HSCT have been also showing poor result in MLD with unpredictable outcomes which confirmed largely ineffective in late infant onset patients. This highlights the need for innovative therapeutic approaches in this field.

### 3. SUMMARY

Provided herein are lentiviral vectors carrying the ARSA gene cassette to induce ARSA expression from stem cells. In one embodiment, the vectors are used in a clinical trial of gene therapy for MLD. The disclosed vectors achieved efficient induction of ARSA. In certain embodiments, the gene expression activity of the vector was assessed at the mRNA and protein levels. In certain embodiments, the effect of ARSA expression on the patients was characterized.

Provided herein are vectors that efficiently induce ARSA genes and produce sufficient levels of an ARSA to improve the physiological parameters of MLD and are utilized for clinical gene therapy.

One embodiment of the present disclosure is a recombinant lentiviral vector (LV) comprising an expression cassette comprising a nucleic acid construct comprising an ARSA gene encoding an ARSA polypeptide. In one embodiment, the LV is a self-inactivating (SIN) lentiviral vector.

In certain embodiments, the vector comprises a recombinant ARSA gene under the control of the ARSA gene 5′ promoter and the ARSA 3′ enhancer. In one embodiment, the recombinant ARSA gene includes exons and introns. In one embodiment, the vector does not comprise ARSA gene introns.

In one embodiment, the vector comprises an insulator.

In one embodiment, the vector comprises a Rev Responsive Element (RRE).

In one embodiment, the vector comprises a central polypurine tract.

In one embodiment, the vector comprises a post-translational regulatory element.

In one embodiment, the posttranscriptional regulatory element is a modified Woodchuck Post-transcriptional Regulatory Element (“WPRE”).

In certain embodiment, the modified WPRE has a nucleic acid sequence as set forth in SEQ ID NO: 3.

In certain embodiments of the vector, the ARSA gene is human ARSA gene. In certain embodiments, the ARSA gene has a nucleic acid sequence as set forth in SEQ ID NO: 2.

A further embodiment of the present disclosure is a host cell transduced with the vector.

In certain embodiments, the cell is a stem cell.

In certain embodiments, the cell is an autologous stem cell.

In certain embodiments, the cell is a hematopoietic stem cell.

Provide herein is a method of growing lentivirus comprising the vectors in a media provided herein. Also provided is a media for suitable for cryopreservation of the lentivirus.

Provided herein is a method of culturing stem cells comprising the vector disclosed herein wherein the stem cells maintained multipotency. In certain embodiments, the media comprises TPO, SCF, FLT3, and IL-3 factors. In certain embodiments, the media comprises kinase inhibitors that inhibit mTOR and ROCK activities. In certain embodiments, the HSCs in the disclosed cultural medium maintained multipotency and increased sensitivity for LV infection.

In certain embodiments, the culture media maintained multipotency of the stem cells and comprise CP1, glucose, Albumin at 2-20% (or 5-10%) and herceptin. In one embodiment, the stem cell is monocyte stem cell.

Disclosed herein is a method of treating metachromatic leukodystrophy (MLD) in a subject, said method comprising transplanting stem cells to the subject comprising a vector expressing the ARSA gene.

Also provided is a method of maintaining the multipotency and increased sensititivity for LV infection of the host cell as described herein comprising incubating the host cells in a media comprising TPO, SCF, FLT3, and IL-3 factors.

In one embodiment, the media further comprises kinase inhibitors that inhibit mTOR and ROCK activity.

In one embodiment, the media comprises CP1, glucose, Albumin at 2-20% and Herceptin.

In one embodiment, the albumin is at 5-10%.

In one embodiment, the media is suitable for cryopreservation of the stem cells.

Provided herein is a method of treating Metachromatic leukodystrophy (MLD) in a subject, said method comprising: transducing a stem cell from said subject with a vector described herein; and transplanting said transduced cell into said subject where said cells express said ARSA gene.

In one embodiment, the cell is a stem cell derived from bone marrow.

In one embodiment, the cell is a human hematopoietic stem or progenitor cell.

Provided herein is a method of regulating expression of a transgene comprising delivering the vector described herein to a hematopoietic stem or progenitor cell.

In one embodiment, the method increases the chances of survival of the hematopoietic stem or progenitor cell during gene therapy.

In one embodiment, the method prevents apoptosis of the hematopoietic stem or progenitor cell.

Provided herein is a pharmaceutical composition comprising the vector expressing the ARSA gene, for treating metachromatic leukodystrophy (MLD) in a subject.

### 4. BRIEF DESCRIPTION OF THE DRAWINGS

For the purpose of illustrating the invention, there are depicted in drawings certain embodiments of the invention. However, the invention is not limited to the precise arrangements and instrumentalities of the embodiments depicted in the drawings.

FIG. 1 shows a vector map that may be used in the present disclosure. To manufacture a vector designed for LV-ARSA production, a lentiviral plasmid back bone AB.PCCL.sin.cPPT.u6miR-10-decoy.hpgk.gfp.wpre is purchased from Addgene (Addgene plasmid #46602; http://n2t.net/addgene:46602; RRID:Addgene_46602). u6-miR-10-Decoy was removed with BsmBI and the vector is re-ligated. GFP is then removed with AgeI and Salt (IDF) SalI-WPRE-SacII was subcloned by PCR into site directed mutagenesis (SDM) vector. WPRE is mutated by replacing ATG to AGG; SDM vector is now SalI-WPRE-mutated-SacII. hARSA cDNA is subcloned by PCR into SDM WPRE.muta1. SDM vector is then ageI-hARSA-salI-WPRE.mut1-sacII. GFP.WPRE cassette is subsequently replaced with hARSA.WPRE.mut1 cassette using AgeI and SacII digest. (IDF)

FIG. 2 shows A schematic diagram of the procedures of hematopoietic stem cells (HSC) gene modification and HSC transplantation. First, after gene design and laboratory scale of manufacturing is optimized, mass lentiviral production carrying function ARSA genes was outsourced for manufacturing under current-GMP condition. Second, the HSC is mobilized and purified with CD34 positive selection. Third, HSC was infected with lentivirus carrying function ARSA gene. Fourth, genetically modified HSC was frozen down and samples were sent to QC and QA testing. Fifth, patient was recruited to accept bone marrow ablation with chemotherapy to wipe out old HSC. Sixth genetically modified HSC was infused into patient to reconstruct bone marrow. Finally, patient as followed up for at least 3 years.

FIG. 3 is a schematic timeline of HSCGT. The first time of HSC collection was performed 60 days before patient was recruited for HSCGT. The aim of fuirst time of HSC collection is for HSC back up. In case HSGT fails, autologous HSC can be infused back to save patient's life. The second time of HSC collection is for CD34 purification and genetically modification. The processing time is about 3 weeks before QC and QA reports were received. Finally, patient was recruited for chemo-conditioning and wipe-out of old HSC with ARSA gene defects. patient was infused genetically modified HSC and follow up.

FIGS. 4A-D (A-C) show brain scan of a post-transplantation subject: A. Healthy control; B. Before HSCGT; C. Post HSCGT 7 months; D. ARSA activity over time.

FIGS. 5A-D show brain scan of a subject 1 year post-transplantation. The lesion areas are stabilized and no expansion of lesion was observed.

FIG. 6 shows Motor/IQ Scores of the treated MLD patients. The MLD patient presents sharply decreased Motor/IQ before the gene therapy on Sep. 30, 2014. After the treatment, the Motor/IQ of the patient is gradually recovered in two years.

FIGS. 7A-B show integration sites of LV in genome by LAM PCR. The insertion sites are 894 in the whole genome.

FIGS. 8A-C show A. Insertion sites are generally distributed through whole genome. No biased insertion preference was observed; B. Grouping of insertion sites of LV in genome. Although insertion sites were mainly found in the region of protein-coding gene, ncRNA and pseudo, only ten of them were inserted into the promoter and Exon of functional gene; C. GO analysis of all of detected insertion sites that may affect cell biology.

FIGS. 9 A-C show transplantation procedure and result; 10 months after transplantation there is no increased risk of tumorigenesis in NSG SCID mice. A. Schematic picture showing irradiation-based bone marrow ablation following genetically modified CD34 human HSC transplantation (300,000 HSC per mouse); B. Genetically modified CD34 HSC shown normal CFU formation and differentiated into red blood cells and monocytes; C. After 10 months post transplantation, there are around 30% of peripheral blood in NSG SCID mice contain human Cd45 positive monocytes.

FIG. 10 shows ARSA enzyme activity in peripheral blood. ARSA activity was increased to normal level in all three patients.

FIG. 11 shows nucleic acid sequence of plasmid clone 8-SEQ ID NO: 1. First shaded region shows the ARSA gene (SEQ ID NO:2). Second shaded region is WPRE mut 1 (“t” to “G” mutation site in BOLD) (SEQ ID NO: 3). Third shaded region is LTR (SEQ ID NO: 4).

FIGS. 12 A-B show changes in Vector Copy Number (“VCN”) over time: A. Change in PBMNC; B. VCN changes with CD3, CD14, CD15, and CD19 over time.

### 5. DETAILED DESCRIPTION

Hematopoietic stem cells gene transplantation of (HSCGT), which utilizes lentiviral-mediated ARSA overexpression in autologous HSC following re-infusion has emerged as a potentially attractive therapy. In this therapy, HSC is isolated, genetically modified, and returned to the patient as an autologous transplant. The corrected HSCs express high level of functional ARSA, with the added benefit of being able to monitor the lentiviral-mediated ARSA gene insertion sites in host genome. Long-term benefit requires the transplantation of a sufficiently high number of gene-modified HSC, which can repopulate the conditioned bone marrow (“BM”), giving rise to corrected therapeutic blood cells of all hematopoietic lineages. This is quantitatively better source of functional enzyme than normal donor cells when transplanted into patients, because corrected HSCs can pass the blood brain barrier (“BBB”) and induce microglia cells to secrete enough functional ARSA enzyme that is taken up by the recipient neural cells that are ARSA-deficient. Self-renewal of HSC ensures to maintain efficacy for longer period than previously attempted treatment. Autologous transplantation is also associated with significantly reduced transplant-related morbidity, avoiding the risk of severe immune rejection.

Efficient long-term gene modification of HSC and their progeny requires a technology which permits stable integration of the corrective DNA into the genome, without affecting HSC function. The most efficient delivery systems are viral vectors. Sensitive molecular tools have been developed to retrieve vector integration sites with high efficiency of several thousands of integrants per patient, and monitor the engrafted gene-corrected stem cells and their daughter cells.

Safety is the major concern of this approach because viral vectors integrate into the host genome and may bring risk to cause mutagenesis of cancer-related genes or oncogenes. The new generation of modified self-inactive lentiviral-based HSCGT has dramatically increased safety with no evidence of increased risk of cancer in clinical trials after more than 5 years of follow-up. Subsequent to recent clinical trials only in late infants with early onset MLD, more researchers are aiming to cure MLD patients who had progressed to a middle stage of MLD.

### 5.1 Definitions

The terms used in this specification generally have their ordinary meanings in the art, within the context of this invention and the specific context where each term is used. Certain terms are discussed below, or elsewhere in the specification, to provide additional guidance to the practitioner in describing the methods of the invention and how to use them. Moreover, it will be appreciated that the same thing can be said in more than one way. Consequently, alternative language and synonyms may be used for any one or more of the terms discussed herein, nor is any special significance to be placed upon whether or not a term is elaborated or discussed herein. Synonyms for certain terms are provided. A recital of one or more synonyms does not exclude the use of the other synonyms. The use of examples anywhere in the specification, including examples of any terms discussed herein, is illustrative only, and in no way limits the scope and meaning of the invention or any exemplified term. Likewise, the invention is not limited to its preferred embodiments.

Hematopoietic stem cells (HSCs) are multipotent stem cells that give rise to all the blood cell types including myeloid (monocytes and macrophages, neutrophils, basophils, eosinophils, erythrocytes, megakaryocytes/platelets, dendritic cells), and lymphoid lineages (T-cells, B-cells, NK-cells). (Biffi, 2018)

“Recombinant” is used consistently with its usage in the art to refer to a nucleic acid sequence that comprises portions that do not naturally occur together as part of a single sequence or that have been rearranged relative to a naturally occurring sequence. A recombinant nucleic acid is created by a process that involves the hand of man and/or is generated from a nucleic acid that was created by hand of man (e.g., by one or more cycles of replication, amplification, transcription, etc.). A recombinant virus is one that comprises a recombinant nucleic acid. A recombinant cell is one that comprises a recombinant nucleic acid. (Kohn, US2015/00224209)

As used herein, the term “recombinant lentiviral vector” or “recombinant LV” refers to an artificially created polynucleotide vector assembled from an LV and a plurality of additional segments as a result of human intervention and manipulation. (Kohn, US2015/00224209)

The phrase “therapeutically effective amount” is used herein to mean an amount sufficient to cause an improvement in a clinically significant condition in the subject, or delays or minimizes or mitigates one or more symptoms associated with the disease, or results in a desired beneficial change of physiology in the subject.

The terms “treat”, “treatment”, and the like refer to a means to slow down, relieve, ameliorate or alleviate at least one of the symptoms of the disease, or reverse the disease after its onset.

The term “subject” as used in this application means an animal with an immune system such as avians and mammals. Mammals include canines, felines, rodents, bovine, equines, porcines, ovines, and primates. Avians include, but are not limited to, fowls, songbirds, and raptors. Thus, the invention can be used in veterinary medicine, e.g., to treat companion animals, farm animals, laboratory animals in zoological parks, and animals in the wild. The invention is particularly desirable for human medical applications.

The term “in need thereof” would be a subject known or suspected of having or being at risk of developing a disease including but not limited to multiple sclerosis and other T cell related autoimmune diseases, or diseases related to the central nervous system or the blood-brain barrier or the blood-spinal cord barrier.

A subject in need of treatment would be one that has already developed the disease. A subject in need of prevention would be one with risk factors of the disease.

The term “about” when refer to a numerical value means±0.5% of the numerical value.

The term “agent” as used herein means a substance that produces or is capable of producing an effect and would include, but is not limited to, chemicals, pharmaceuticals, drugs, biologics, small molecules, antibodies, nucleic acids, peptides, and proteins.

### 5.2 Gene Vector

The ARSA gene or other genes that are useful in the present disclosure may be expressed with a suitable gene vector, i.e. a vector suitable for delivering a gene (transgene) of interest, such as a viral vector. Viral vectors suitable for gene therapy are well known in the art. Viruses from several different families have been modified to generate viral vectors for gene delivery. Viruses which can be used in the present disclosure include retroviruses, lentivirus, adenoviruses, adeno-associated viruses, herpes simplex viruses, picornaviruses, and alphaviruses. In one embodiment, the virus is a retrovirus. In one embodiment, the retrovirus is lentivirus. The present disclosure can be used to control expression of a transgene included in the vector. The invention can also be used to control expression of the vector. In one embodiment, the vector disclosed herein may be used to deliver one or more transgenes useful in the treatment of disorders as described in the present disclosure. The delivery of one or more therapeutic genes by the vector system according to the disclosure may be used alone or in combination with other treatment or agents for the treatment.

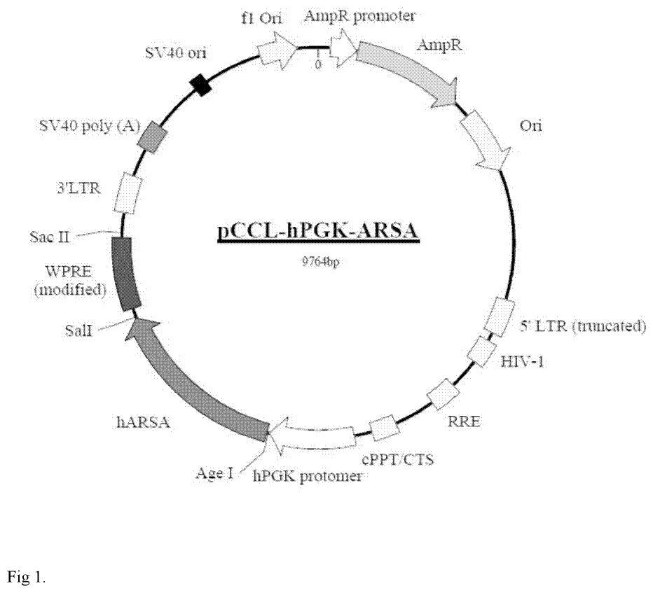

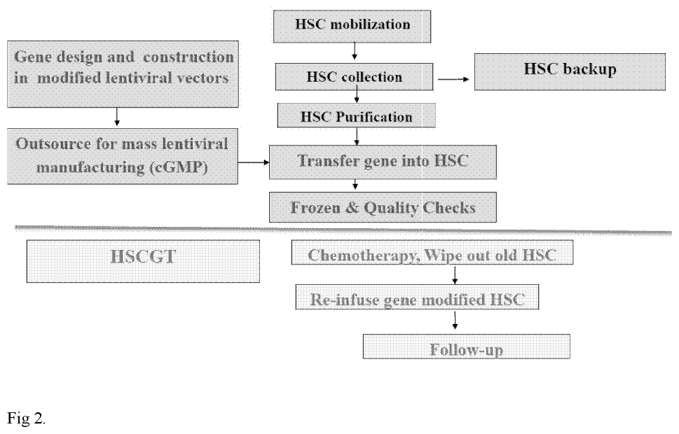

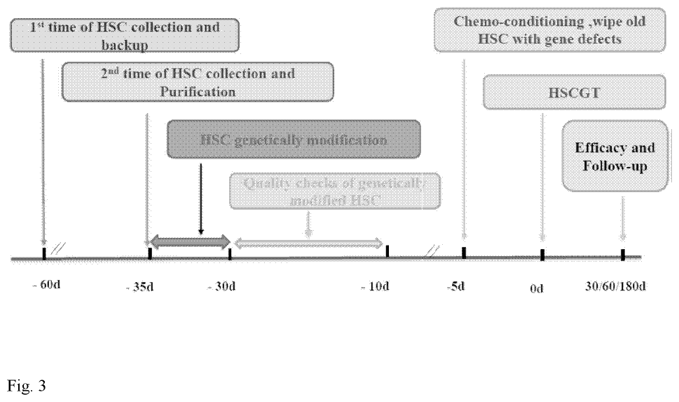

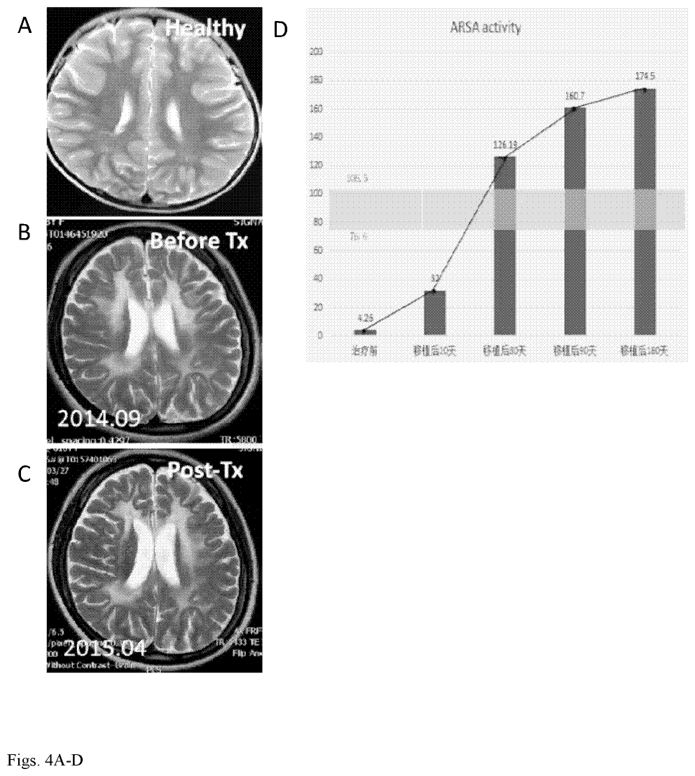

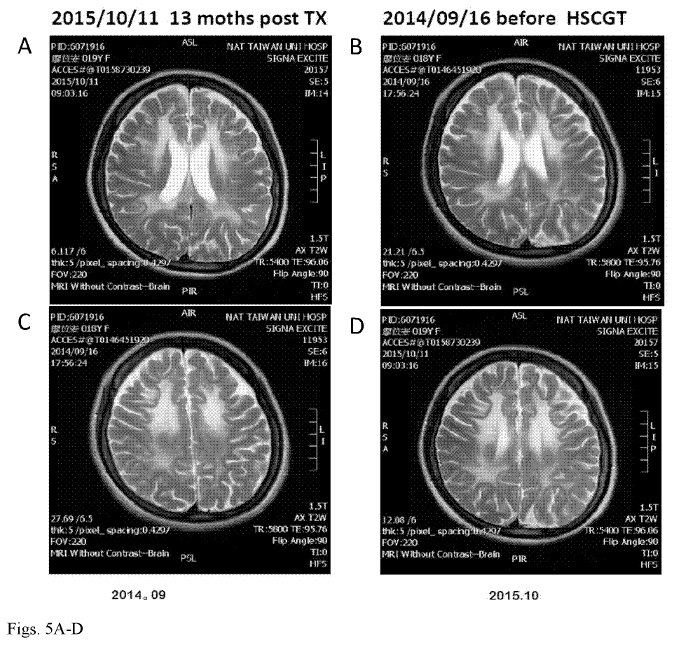

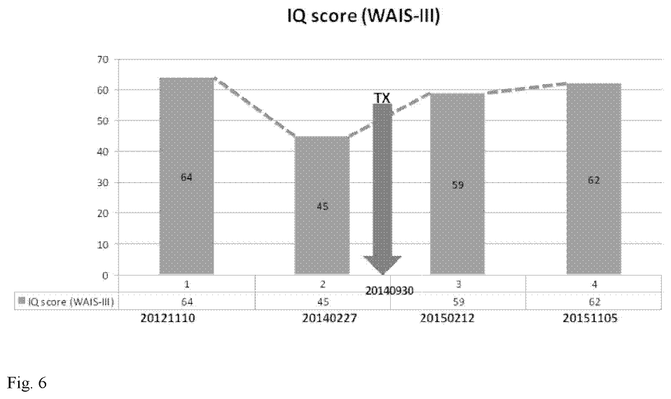

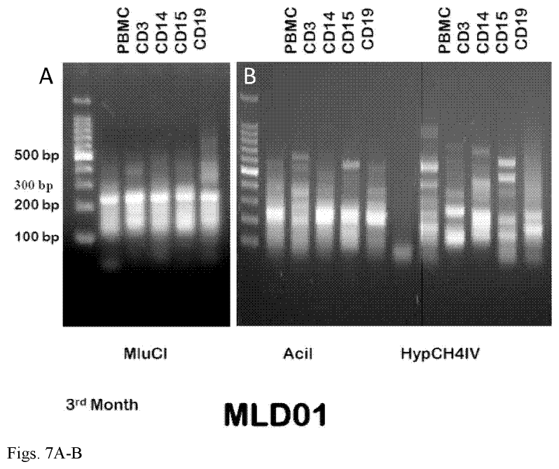

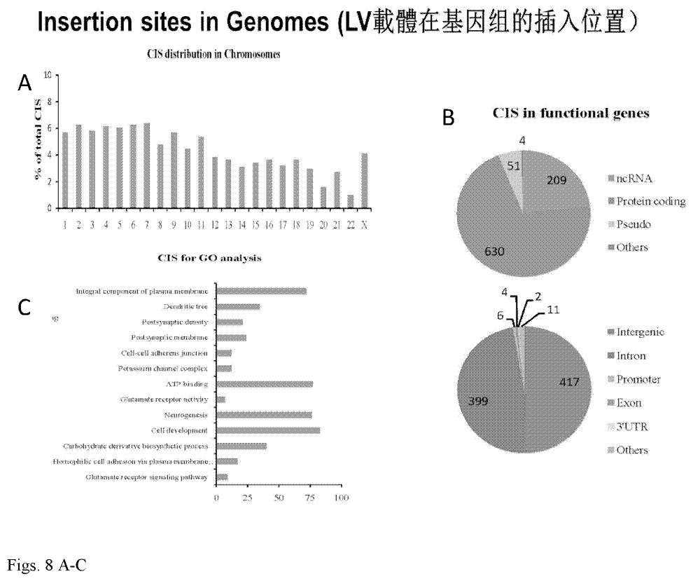

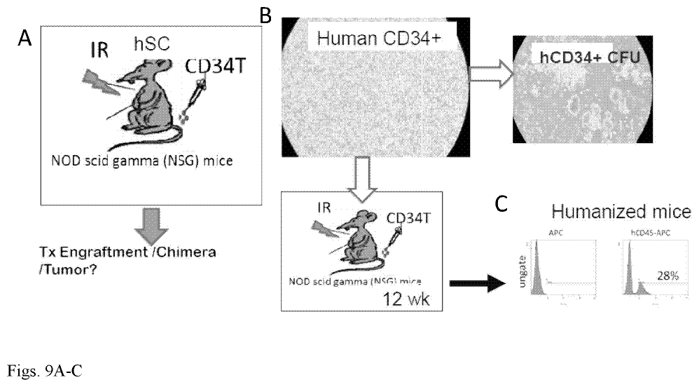

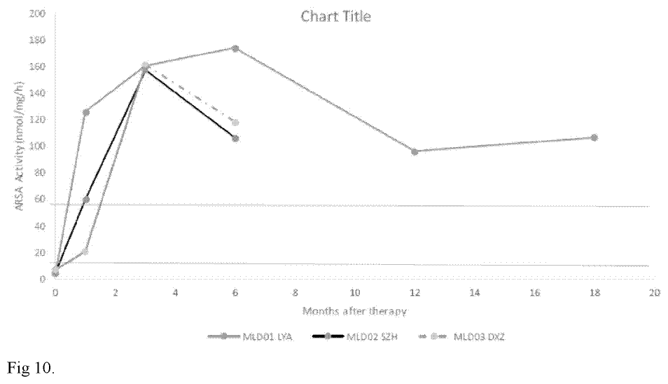

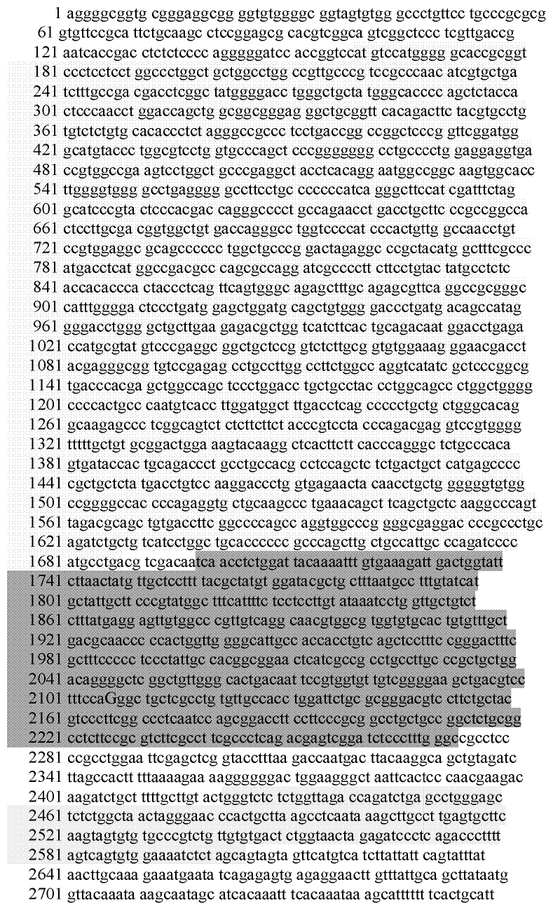

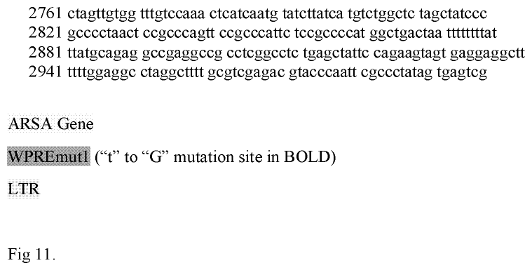

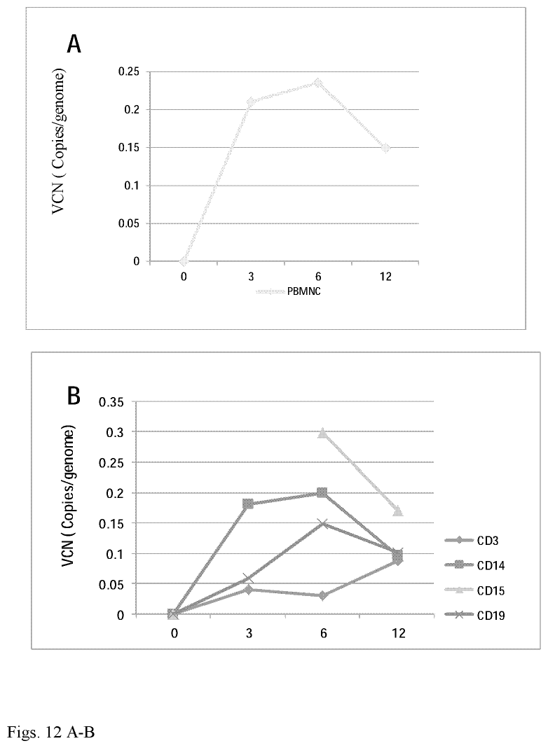

In one embodiment, a backbone vector that is commercially available AB.PCCL.sin.cPPTu6miR-10-deoy.hpgk.gfp.wpre may be used. In one embodiment, u6-miR-10-Decoy is removed from the backbone with BsmBI and relegate the vector. In one embodiment, GFP is removed using AgeI/SalI. SalI-WPRE-SacII was subcloned into SDM vector using PCR. In one embodiment, wild type ATG is mutated to AGG to obtained WPRE mutation. In one embodiment, the vector is SDM Sal I-WPRE mutated-SacII. In one embodiment, an hARSA cDNA is subcloned into SDM WPRE.muta1 via PCT. In one embodiment, the vector is SDM vector: AgeI-hARSA-SalI-WPRE mut 1-SacII. In one embodiment, the GFP.WPRE cassette is replaced with hARSA.WPRE mut 1 cassette using AgeI/SacII digest. In one embodiment, the transgene vector for LV product is transfected in 293FT with MDL/VSV.G. In one embodiment, the vector is shown in FIG. 1.

### 5.2.1 TAT-Independent and Self Inactivating Lentiviral Vectors

In certain embodiments, the LV constructs comprise a TAT-independent, self-inactivating (SIN) configuration. Thus, in various embodiments it is desirable to employ in the LVs described herein an LTR region that has reduced promoter activity relative to wild-type LTR. Such constructs can be provided that are effectively “self-inactivating” (SIN) which provides a biosafety feature. SIN vectors are ones in which the production of full-length vector RNA in transduced cells is greatly reduced or abolished altogether. This feature minimizes the risk that replication-competent recombinants (RCRs) will emerge. Furthermore, it reduces the risk that that cellular coding sequences located adjacent to the vector integration site will be aberrantly expressed.

Furthermore, a SIN design reduces the possibility of interference between the LTR and the promoter that is driving the expression of the transgene. SIN LVs can often permit full activity of the internal promoter.

The SIN design increases the biosafety of the LVs. The majority of the HIV LTR is comprised of the U3 sequences. The U3 region contains the enhancer and promoter elements that modulate basal and induced expression of the HIV genome in infected cells and in response to cell activation. Several of these promoter elements are essential for viral replication. Some of the enhancer elements are highly conserved among viral isolates and have been implicated as critical virulence factors in viral pathogenesis. The enhancer elements may act to influence replication rates in the different cellular target of the virus.

As viral transcription starts at the 3′ end of the U3 region of the 5′ LTR, those sequences are not part of the viral mRNA and a copy thereof from the 3′ LTR acts as template for the generation of both LTR's in the integrated provirus. If the 3′ copy of the U3 region is altered in a retroviral vector construct, the vector RNA is still produced from the intact 5′ LTR in producer cells, but cannot be regenerated in target cells. Transduction of such a vector results in the inactivation of both LTR's in the progeny virus. Thus, the retrovirus is self-inactivating (SIN) and those vectors are known as SIN transfer vectors.

In certain embodiments self-inactivation is achieved through the introduction of a deletion in the U3 region of the 3′ LTR of the vector DNA, i.e., the DNA used to produce the vector RNA. During RT, this deletion is transferred to the 5′ LTR of the proviral DNA. Typically, it is desirable to eliminate enough of the U3 sequence to greatly diminish or abolish altogether the transcriptional activity of the LTR, thereby greatly diminishing or abolishing the production of full-length vector RNA in transduced cells. However, it is generally desirable to retain those elements of the LTR that are involved in polyadenylation of the viral RNA, a function typically spread out over U3, R and U5. Accordingly, in certain embodiments, it is desirable to eliminate as many of the transcriptionally important motifs from the LTR as possible while sparing the polyadenylation determinants.

The SIN design is described in detail in Zufferey et al. (1998) J Virol. 72(12): 9873-9880, and in U.S. Pat. No. 5,994,136. As described therein, there are, however, limits to the extent of the deletion at the 3′ LTR. First, the 5′ end of the U3 region serves another essential function in vector transfer, being required for integration (terminal dinucleotide+att sequence). Thus, the terminal dinucleotide and the att sequence may represent the 5′ boundary of the U3 sequences which can be deleted. In addition, some loosely defined regions may influence the activity of the downstream polyadenylation site in the R region. Excessive deletion of U3 sequence from the 3′LTR may decrease polyadenylation of vector transcripts with adverse consequences both on the titer of the vector in producer cells and the transgene expression in target cells.

In certain embodiments, the lentiviral sequences removed from the LTRs are replaced with comparable sequences from a non-lentiviral retrovirus, thereby forming hybrid LTRs. In particular, the lentiviral R region within the LTR can be replaced in whole or in part by the R region from a non-lentiviral retrovirus. In certain embodiments, the lentiviral TAR sequence, a sequence which interacts with TAT protein to enhance viral replication, is removed, preferably in whole, from the R region. The TAR sequence is then replaced with a comparable portion of the R region from a non-lentiviral retrovirus, thereby forming a hybrid R region. The LTRs can be further modified to remove and/or replace with non-lentiviral sequences all or a portion of the lentiviral U3 and U5 regions.

In certain embodiments, the SIN configuration provides a retroviral LTR comprising a hybrid lentiviral R region that lacks all or a portion of its TAR sequence, thereby eliminating any possible activation by TAT, wherein the TAR sequence or portion thereof is replaced by a comparable portion of the R region from a non-lentiviral retrovirus, thereby forming a hybrid R region. In one embodiment, the retroviral LTR comprises a hybrid R region, wherein the hybrid R region comprises a portion of the HIV R. Suitable lentiviruses from which the R region can be derived include, for example, HIV (HIV-1 and HIV-2), EIV, SIV and FIV. Suitable retroviruses from which non-lentiviral sequences can be derived include, for example, MoMSV, MoMLV, Friend, MSCV, RSV and Spumaviruses. In one embodiment, the lentivirus is HIV and the non-lentiviral retrovirus is MoMSV.

In one embodiment, the LTR comprising a hybrid R region is a left (5′) LTR and further comprises a promoter sequence upstream from the hybrid R region. In certain embodiments, promoters are non-lentiviral in origin and include, for example, the U3 region from a non-lentiviral retrovirus (e.g., the MoMSV U3 region). In one embodiment, the left (5′) LTR further comprises a lentiviral U5 region downstream from the hybrid R region. In one embodiment, the U5 region is the HIV U5 region including the HIV att site necessary for genomic integration.

In one embodiment, the LTR comprising a hybrid R region is a right (3′) LTR and further comprises a modified (e.g., truncated) lentiviral U3 region upstream from the hybrid R region. The modified lentiviral U3 region can include the att sequence, but lack any sequences having promoter activity, thereby causing the vector to be SIN in that viral transcription cannot go beyond the first round of replication following chromosomal integration. In a particular embodiment, the modified lentiviral U3 region upstream from the hybrid R region consists of the 3′ end of a lentiviral U3 region up to and including the lentiviral U3 att site.

In certain embodiments, the vectors tolerate significant U3 deletions, including the removal of the LTR TATA box (e.g., deletions from −418 to −18), without significant reductions in vector titers. These deletions render the LTR region substantially transcriptionally inactive in that the transcriptional ability of the LTR in reduced to about 90% or lower.

It has also been demonstrated that the trans-acting function of Tat becomes dispensable if part of the upstream LTR in the transfer vector construct is replaced by constitutively active promoter sequences (see, e.g., Dull et al. (1998) J Virol. 72(11): 8463-8471. In one embodiments, the cassette expressing ARSA is placed in the pCCL LV backbone, which is a SIN vector with the CMV enhancer/promoter substituted in the 5′ LTR.

It will be recognized that the CMV promoter typically provides a high level of non-tissue specific expression. Other promoters with similar constitutive activity include, but are not limited to the RSV promoter, and the SV40 promoter. Mammalian promoters such as the beta-actin promoter, ubiquitin C promoter, elongation factor promoter, tubulin promoter, etc., may also be used.

The foregoing SIN configurations are illustrative and non-limiting. Numerous SIN configurations are known to those of skill in the art. As indicated above, in certain embodiments, the LTR transcription is reduced by about 95% to about 99%. In certain embodiments LTR may be rendered at least about 90%, at least about 91%, at least about 92%, at least about 93%, at least about 94%, at least about 95% at least about 96%, at least about 97%, at least about 98%, or at least about 99% transcriptionally inactive.

### Insulator Element

In certain embodiments, to further enhance biosafety, insulators are inserted into the LV described herein. Insulators are DNA sequence elements present throughout the genome. They bind proteins that modify chromatin and alter regional gene expression. The placement of insulators in the vectors described herein offer various potential benefits including, inter alia: 1) Shielding of the vector from positional effect variegation of expression by flanking chromosomes (i.e., barrier activity); and 2) Shielding flanking chromosomes from insertional trans-activation of gene expression by the vector (enhancer blocking). Thus, insulators can help to preserve the independent function of genes or transcription units embedded in a genome or genetic context in which their expression may otherwise be influenced by regulatory signals within the genome or genetic context (see, e.g., Burgess-Beusse et al. (2002) Proc. Natl. Acad. Sci. USA, 99: 16433; and Zhan et al. (2001) Hum. Genet., 109: 471). In the present context insulators may contribute to protecting lentivirus-expressed sequences from integration site effects, which may be mediated by cis-acting elements present in genomic DNA and lead to deregulated expression of transferred sequences. In various embodiments LVs are provided in which an insulator sequence is inserted into one or both LTRs or elsewhere in the region of the vector that integrates into the cellular genome.

In various embodiments, the vectors described herein further comprise a packaging signal. A “packaging signal,” “packaging sequence,” or “psi sequence” is any nucleic acid sequence sufficient to direct packaging of a nucleic acid whose sequence comprises the packaging signal into a retroviral particle. The term includes naturally occurring packaging sequences and also engineered variants thereof. Packaging signals of a number of different retroviruses, including lentiviruses, are known in the art.

In certain embodiments the LVs described herein comprise a Rev response element (RRE) to enhance nuclear export of unspliced RNA. RREs are well known to those of skill in the art. Such sequences are readily available from Genbank or from the database with URL hiv-web.lanl.gov/content/index.

In various embodiments the vectors described herein further include a central polypurine tract. Insertion of a fragment containing the central polypurine tract (cPPT) in lentiviral (e.g., HIV-1) vector constructs is known to enhance transduction efficiency drastically, reportedly by facilitating the nuclear import of viral cDNA through a central DNA flap.

In certain embodiments the LVs described herein may comprise any of a variety of posttranscriptional regulatory elements (PREs) whose presence within a transcript increases expression of the heterologous nucleic acid (e.g., βAS3) at the protein level. PREs may be particularly useful in certain embodiments, especially those that involve lentiviral constructs with modest promoters.

One type of PRE is an intron positioned within the expression cassette, which can stimulate gene expression. However, introns can be spliced out during the life cycle events of a lentivirus. Hence, if introns are used as PRE's they are typically placed in an opposite orientation to the vector genomic transcript.

Posttranscriptional regulatory elements that do not rely on splicing events offer the advantage of not being removed during the viral life cycle. Some examples are the posttranscriptional processing element of herpes simplex virus, the posttranscriptional regulatory element of the hepatitis B virus (HPRE) and the woodchuck hepatitis virus (WPRE). Of these the WPRE is typically preferred as it contains an additional cis-acting element not found in the HPRE. This regulatory element is typically positioned within the vector so as to be included in the RNA transcript of the transgene, but outside of stop codon of the transgene translational unit.

WPRE is an RNA export element that mediates efficient transport of RNA from the nucleus to the cytoplasm. It enhances the expression of transgenes by insertion of a cis-acting nucleic acid sequence, such that the element and the transgene are contained within a single transcript. Presence of the WPRE in the sense orientation was shown to increase transgene expression by up to 7 to 10 fold. Retroviral vectors transfer sequences in the form of cDNAs instead of complete intron-containing genes as introns are generally spliced out during the sequence of events leading to the formation of the retroviral particle. Introns mediate the interaction of primary transcripts with the splicing machinery. Because the processing of RNAs by the splicing machinery facilitates their cytoplasmic export, due to a coupling between the splicing and transport machineries, cDNAs are often inefficiently expressed. Thus, the inclusion of the WPRE in a vector results in enhanced expression of transgenes.

The woodchuck hepatitis virus posttranscriptional regulatory element (WPRE) is a hepadnavirus sequence that is widely used as a cis-acting regulatory module in various types of plasmid or viral gene vectors. The WPRE is often essential to achieve sufficient levels of expression with integrative retroviral vectors configured for gene therapy; therefore, its mechanisms of action have been studied in detail. When placed in the 30 untranslated region of gene transfer cassettes, the WPRE enhances the expression of the transgene by increasing both nuclear and cytoplasmic mRNA levels, early during the biogenesis of RNA transcripts. (Zufferey R, Donello J E, Trono D, Hope T J. Woodchuck hepatitis virus posttranscriptional regulatory element enhances expression of transgenes delivered by retroviral vectors. J Virol 1999; 73: 2886-2892) (Higashimoto T, Urbinati F, Perumbeti A, Jiang G, Zarzuela A, Chang L J et al. The woodchuck hepatitis virus post-transcriptional regulatory element reduces readthrough transcription from retroviral vectors. Gene Therapy 2007; 14: 1298-1304.)

Although the WPRE is coupled in part to the CRM1-dependent export machinery, its posttranscriptional effects do not result from increased RNA export or from an increased rate of transcription or longer half-life of RNA species but rather from improved 30 end transcript processing. (Popa I, Harris M E, Donello J E, Hope T J. CRM1-dependent function of a cis-acting RNA export element. Mol Cell Biol 2002; 22: 2057-2067.) The WPRE seems to increase the amount of polyadenylated transcripts and clearly augments the size of the polyA tail of RNA. (Schambach A, Galla M, Maetzig T, Loew R, Baum C. Improving transcriptional termination of self-inactivating gamma-retroviral and lentiviral vectors. Mol Ther 2007; 15: 1167-1173.) The polyA motifs are contained in the long terminal repeats (LTRs) of gammaretroviral or of human immunodeficiency virus (HIV)-1-derived gene transfer cassettes. When placed upstream of the 30 LTR, the WPRE improves transcript termination and therefore can significantly reduce transcript read-through, particularly with Moloney-derived vectors. (Higashimoto et al. 2007 Gene Therapy 14:1298-1304) (Schambach, et al. 2007 Mol Ther 15:1167-1173).

### 5.3 Transduced Host Cells and Methods of Cell Transduction

The recombinant LV and resulting virus described herein are capable of transferring a nucleic acid (e.g., a nucleic acid encoding an ARSA gene sequence into a mammalian cell. For delivery to cells, vectors of the present invention are preferably used in conjunction with a suitable packaging cell line or co-transfected into cells in vitro along with other vector plasmids containing the necessary retroviral genes (e.g., gag and pol) to form replication incompetent virions capable of packaging the vectors of the present invention and infecting cells.

The recombinant LVs and resulting virus described herein are capable of transferring a nucleic acid (e.g., a nucleic acid encoding ARSA) sequence into a mammalian cell. For delivery to cells, vectors of the present invention are preferably used in conjunction with a suitable packaging cell line or co-transfected into cells in vitro along with other vector plasmids containing the necessary retroviral genes (e.g., gag and pol) to form replication incompetent virions capable of packaging the vectors of the present invention and infecting cells.

Typically, the vectors are introduced via transfection into the packaging cell line. The packaging cell line produces viral particles that contain the vector genome. Methods for transfection are well known by those of skill in the art. After cotransfection of the packaging vectors and the transfer vector to the packaging cell line, the recombinant virus is recovered from the culture media and tittered by standard methods used by those of skill in the art. Thus, the packaging constructs can be introduced into human cell lines by calcium phosphate transfection, lipofection or electroporation, generally together with a dominant selectable marker, such as neomycin, DHFR, Glutamine synthetase, followed by selection in the presence of the appropriate drug and isolation of clones. In certain embodiments the selectable marker gene can be linked physically to the packaging genes in the construct.

Stable cell lines wherein the packaging functions are configured to be expressed by a suitable packaging cell are known. In general, for the production of virus particles, one may employ any cell that is compatible with the expression of lentiviral Gag and Pol genes, or any cell that can be engineered to support such expression. For example, producer cells such as 293T cells and HT1080 cells may be used.

The packaging cells with a lentiviral vector incorporated in them form producer cells. Producer cells are thus cells or cell-lines that can produce or release packaged infectious viral particles carrying the therapeutic gene of interest (e.g. ARSA). These cells can further be anchorage dependent which means that these cells will grow, survive, or maintain function optimally when attached to a surface such as glass or plastic. Some examples of anchorage dependent cell lines used as lentiviral vector packaging cell lines when the vector is replication competent are HeLa or 293 cells and PERC.6 cells.

Accordingly, in certain embodiments, methods are provided of delivering a gene to a cell which is then integrated into the genome of the cell, comprising contacting the cell with a virion containing a lentiviral vector described herein. The cell (e.g., in the form of tissue or an organ) can be contacted (e.g., infected) with the virion ex vivo and then delivered to a subject (e.g., a mammal, animal or human) in which the gene (e.g., ARSA) will be expressed. In various embodiments the cell can be autologous to the subject (i.e., from the subject) or it can be non-autologous (i.e., allogeneic or xenogenic) to the subject. Moreover, because the vectors described herein are capable of being delivered to both dividing and non-dividing cells, the cells can be from a wide variety including, for example, bone marrow cells, mesenchymal stem cells (e.g., obtained from adipose tissue), and other primary cells derived from human and animal sources. Alternatively, the virion can be directly administered in vivo to a subject or a localized area of a subject (e.g., bone marrow).

Of course, as noted above, the lentivectors described herein will be particularly useful in the transduction of human hematopoietic progenitor cells or a hematopoietic stem cells, obtained either from the bone marrow, the peripheral blood or the umbilical cord blood.

### 5.4 Gene Therapy

In still other embodiments, the present invention is directed to a method for transducing a human hematopoietic stem cell comprising contacting a population of human cells that include hematopoietic stem cells with one of the foregoing lentivectors under conditions to effect the transduction of a human hematopoietic progenitor cell in said population by the vector. The stem cells may be transduced in vivo or in vitro, depending on the ultimate application. Even in the context of human gene therapy, such as gene therapy of human stem cells, one may transduce the stem cell in vivo or, alternatively, transduce in vitro followed by infusion of the transduced stem cell into a human subject. In one aspect of this embodiment, the human stem cell can be removed from a human, e.g., a human patient, using methods well known to those of skill in the art and transduced as noted above. The transduced stem cells are then reintroduced into the same or a different human.

### 5.4.1. Stem Cell/Progenitor Cell Gene Therapy

In various embodiments the lentivectors described herein are particularly useful for the transduction of human hematopoietic progenitor cells or hematopoietic stem cells (HSCs), obtained either from the bone marrow, the peripheral blood or the umbilical cord blood. When cells, peripheral blood cells or tumor cells are transduced ex vivo, the vector particles are incubated with the cells using a dose generally in the order of between 1 to 50 multiplicities of infection (MOI) which also corresponds to 1×10⁵ to 50×10⁵ transducing units of the viral vector per 10⁵ cells. This of course includes amount of vector corresponding to 1, 2, 3, 4, 5, 6, 7, 8, 9, 10, 15, 20, 25, 30, 35, 40, 45, and 50 MOI. Typically, the amount of vector may be expressed in terms of HeLa transducing units (TU).

It is noted that, a dose-related increase in gene transfer achieved (the average VC/cell measured by qPCR) was found only for vector concentrations below 2×10⁷ TU/ml. Higher vector concentrations did not increase the transduction efficacy and, in fact, often had a negative effect on the extent of transduction (data not shown). Based on these findings, the CCL-βAS3-FB vector was used at a standard concentration of 2×10⁷ TU/ml (MOI=40).

In certain embodiments cell-based therapies involve providing stem cells and/or hematopoietic precursors, transduce the cells with the lentivirus encoding an ARSA gene and then introduce the transformed cells into a subject in need thereof (e.g., a subject with MLD). In certain embodiments the methods involve isolating population of cells, e.g., stem cells from a subject, optionally expand the cells in tissue culture, and administer the lentiviral vector whose presence within a cell results in production of ARSA in the cells in vitro. The cells are then returned to the subject, where, for example, they may provide ARSA.

In some embodiments, a population of cells, which may be cells from a cell line or from an individual other than the subject, can be used. Methods of isolating stem cells, immune system cells, etc., from a subject and returning them to the subject are well known in the art. Such methods are used, e.g., for bone marrow transplant, peripheral blood stem cell transplant.

Where stem cells are to be used, it will be recognized that such cells can be derived from a number of sources including bone marrow (BM), cord blood (CB) CB, mobilized peripheral blood stem cells (mPB SC), and the like. In certain embodiments the use of induced pluripotent stem cells (HSCs) is contemplated. Methods of isolating hematopoietic stem cells (HSCs), transducing such cells and introducing them into a mammalian subject are well known to those of skill in the art.

### 5.4.2 Direct Introduction of Vector

In certain embodiments direct treatment of a subject by direct introduction of the vector is contemplated. The lentiviral compositions may be formulated for delivery by any available route including, but not limited to parenteral (e.g., intravenous), intradermal, subcutaneous, oral (e.g., inhalation), transdermal (topical), transmucosal, rectal, and vaginal. Commonly used routes of delivery include inhalation, parenteral, and transmucosal.

In various embodiments pharmaceutical compositions can include an LV in combination with a pharmaceutically acceptable carrier. As used herein the language “pharmaceutically acceptable carrier” includes solvents, dispersion media, coatings, antibacterial and antifungal agents, isotonic and absorption delaying agents, and the like, compatible with pharmaceutical administration. Supplementary active compounds can also be incorporated into the compositions.

In some embodiments, active agents, i.e., a lentiviral described herein and/or other agents to be administered together the vector, are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such compositions will be apparent to those skilled in the art. Suitable materials can also be obtained commercially from Alza Corporation and Nova Pharmaceuticals, Inc. Liposomes can also be used as pharmaceutically acceptable carriers. These can be prepared according to methods known to those skilled in the art. In some embodiments the composition is targeted to particular cell types or to cells that are infected by a virus. For example, compositions can be targeted using monoclonal antibodies to cell surface markers, e.g., endogenous markers or viral antigens expressed on the surface of infected cells.

It is advantageous to formulate compositions in dosage unit form for ease of administration and uniformity of dosage. Dosage unit form as used herein refers to physically discrete units suited as unitary dosages for the subject to be treated; each unit comprising a predetermined quantity of a LV calculated to produce the desired therapeutic effect in association with a pharmaceutical carrier.

A unit dose need not be administered as a single injection but may comprise continuous infusion over a set period of time. Unit dose of the LV described herein may conveniently be described in terms of transducing units (T.U.) of lentivector, as defined by titering the vector on a cell line such as HeLa or 293. In certain embodiments unit doses can range from 10³, 10⁴, 10⁵, 10⁶, 10⁷, 10⁸, 10⁹, 10¹⁰, 10¹¹, 10¹², 10¹³ T.U. and higher.

Pharmaceutical compositions can be administered at various intervals and over different periods of time as required, e.g., one time per week for between about 1 to about 10 weeks; between about 2 to about 8 weeks; between about 3 to about 7 weeks; about 4 weeks; about 5 weeks; about 6 weeks, etc. It may be necessary to administer the therapeutic composition on an indefinite basis. The skilled artisan will appreciate that certain factors can influence the dosage and timing required to effectively treat a subject, including but not limited to the severity of the disease or disorder, previous treatments, the general health and/or age of the subject, and other diseases present. Treatment of a subject with a LV can include a single treatment or, in many cases, can include a series of treatments.

Exemplary doses for administration of gene therapy vectors and methods for determining suitable doses are known in the art. It is furthermore understood that appropriate doses of a LV may depend upon the particular recipient and the mode of administration. The appropriate dose level for any particular subject may depend upon a variety of factors including the age, body weight, general health, gender, and diet of the subject, the time of administration, the route of administration, the rate: of excretion, other administered therapeutic agents, and the like.

In certain embodiments lentiviral gene therapy vectors can be delivered to a subject by, for example, intravenous injection, local administration, or by stereotactic injection (see, e.g., Chen et al. (1994) Proc. Natl. Acad. Sci. USA, 91: 3054). In certain embodiments vectors may be delivered orally or inhalation and may be encapsulated or otherwise manipulated to protect them from degradation, enhance uptake into tissues or cells, etc. Pharmaceutical preparations can include a LV in an acceptable diluent, or can comprise a slow release matrix in which a LV is imbedded. Alternatively, or additionally, where a vector can be produced intact from recombinant cells, as is the case for retroviral or lentiviral vectors as described herein, a pharmaceutical preparation can include one or more cells which produce vectors. Pharmaceutical compositions comprising a LV described herein can be included in a container, pack, or dispenser, optionally together with instructions for administration.

### 5.4.3 Hematopoietic Stem Cell Transplantation

A stem cell is able to differentiate into many cell types. A cell that is able to differentiate into all cell types is known as totipotent. In mammals, only the zygote and early embryonic cells are totipotent. Stem cells are cells found in most, if not all, multi-cellular organisms. They are characterized by the ability to renew themselves through mitotic cell division and differentiating into a diverse range of specialized cell types. The two broad types of mammalian stem cells are: embryonic stem cells that are isolated from the inner cell mass of blastocysts, and adult stem cells that are found in adult tissues. In a developing embryo, stem cells can differentiate into all of the specialized embryonic tissues. In adult organisms, stem cells and progenitor cells act as a repair system for the body, replenishing specialized cells, but also maintain the normal turnover of regenerative organs, such as blood, skin or intestinal tissues.

Hematopoietic stem cell transplantation (HSCT) is the transplantation of blood stem cells derived from the bone marrow (in this case known as bone marrow transplantation) or blood. Stem cell transplantation is a medical procedure in the fields of hematology and oncology, most often performed for people with diseases of the blood, bone marrow, or certain types of cancer.

With the availability of the stem cell growth factors GM-CSF and G-CSF, hematopoietic stem cell transplantation procedures may be performed using stem cells collected from the peripheral blood. In one embodiment, the hematopoietic stem cell transplantation is performed using bone marrow. Collecting peripheral blood stem cells provides a bigger graft, does not require that the donor be subjected to general anesthesia to collect the graft, results in a shorter time to engraftment, and may provide for a lower long-term relapse rate.

Hematopoietic stem cell transplantation remains a risky procedure with many possible complications; it has traditionally been reserved for patients with life-threatening diseases. While occasionally used experimentally in nonmalignant and nonhematologic indications such as severe disabling auto-immune disease and cardiovascular disease, the risk of fatal complications appears too high to gain wider acceptance.

Candidates for HSCTs include pediatric cases, children or adults. More recently non-myeloablative, or so-called “mini transplant,” procedures have been developed that require smaller doses of preparative chemo and radiation. This has allowed HSCT to be conducted in the elderly and other patients who would otherwise be considered too weak to withstand a conventional treatment regimen. The present invention aims to widen the therapeutic application of such treatments by improving their safety and/or efficacy.

### 5.5 Culturing HSCs

Following transfection, the cells are plated on feeder culture. The cultures are then fed every 3-4 days with growth medium, which may or may not be supplemented with one or more growth factors. The HSC cell lines can be passaged using standard techniques, such as by trypsinization, when 80-95% confluent. Up to approximately the twentieth passage, it is, in some embodiments, beneficial to maintain selection (by, for example, the addition of G418 for cells containing a neomycin resistance gene). Cells may also be frozen in liquid nitrogen for long-term storage. Clonal cell lines can be isolated as described above. In general, such clonal cell lines may be isolated using standard techniques, such as by limit dilution or using cloning rings, and expanded. Clonal cell lines may generally be fed and passaged as provided in the art. HSC colonies that were transduced with Sendai virus start to form 2 days after plating onto feeder cells. HSC colonies that were transduced with lentivirus start to form 7 days after plating onto feeder cells.

### 5.6 Matrices Comprising HSCs

The disclosure further comprises matrices, hydrogels, scaffolds, and the like that comprise HSCs. HSCs can be seeded onto a natural matrix, e.g., a biomaterial. In certain embodiments, the scaffold is obtained by 3D printing. The HSCs can be suspended in a hydrogel solution suitable for, e.g., injection. Suitable hydrogels for such compositions include self-assembling peptides, such as RAD16. In one embodiment, a hydrogel solution comprising the cells can be allowed to harden, for instance in a mold, to form a matrix having cells dispersed therein for implantation. HSCs in such a matrix can also be cultured so that the cells are mitotically expanded prior to implantation. The hydrogel is, e.g., an organic polymer (natural or synthetic) that is cross-linked via covalent, ionic, or hydrogen bonds to create a three-dimensional open-lattice structure that entraps water molecules to form a gel. Hydrogel-forming materials include polysaccharides such as alginate and salts thereof, peptides, polyphosphazines, and polyacrylates, which are crosslinked ionically, or block polymers such as polyethylene oxide-polypropylene glycol block copolymers which are crosslinked by temperature or pH, respectively. In some embodiments, the hydrogel or matrix of the invention is biodegradable. In some embodiments of the invention, the formulation comprises an in situ polymerizable gel (see., e.g., U.S. Patent Application Publication 2002/0022676; Anseth et al., J. Control Release, 78(1-3):199-209 (2002); Wang et al., Biomaterials, 24(22):3969-80 (2003).

In some embodiments, the polymers are at least partially soluble in aqueous solutions, such as water, buffered salt solutions, or aqueous alcohol solutions, that have charged side groups, or a monovalent ionic salt thereof. Examples of polymers having acidic side groups that can be reacted with cations are poly(phosphazenes), poly(acrylic acids), poly(methacrylic acids), copolymers of acrylic acid and methacrylic acid, poly(vinyl acetate), and sulfonated polymers, such as sulfonated polystyrene. Copolymers having acidic side groups formed by reaction of acrylic or methacrylic acid and vinyl ether monomers or polymers can also be used. Examples of acidic groups are carboxylic acid groups, sulfonic acid groups, halogenated (preferably fluorinated) alcohol groups, phenolic OH groups, and acidic OH groups.

The HSCs or co-cultures thereof can be seeded onto a three-dimensional framework or scaffold and implanted in vivo. Such a framework can be implanted in combination with any one or more growth factors, cells, drugs or other components that stimulate tissue formation or otherwise enhance or improve the practice of the disclosure.

Examples of scaffolds that can be used in the present invention include nonwoven mats, porous foams, or self-assembling peptides. Nonwoven mats can be formed using fibers comprised of a synthetic absorbable copolymer of glycolic and lactic acids (e.g., PGA/PLA) (VICRYL, Ethicon, Inc., Somerville, N.J.). Foams, composed of, e.g., poly(s-caprolactone)/poly(glycolic acid) (PCL/PGA) copolymer, formed by processes such as freeze-drying, or lyophilization (see, e.g., U.S. Pat. No. 6,355,699), can also be used as scaffolds.

The HSCs can also be seeded onto, or contacted with, a physiologically-acceptable ceramic material including, but not limited to, mono-, di-, tri-, alpha-tri-, beta-tri-, and tetra-calcium phosphate, hydroxyapatite, fluoroapatites, calcium sulfates, calcium fluorides, calcium oxides, calcium carbonates, magnesium calcium phosphates, biologically active glasses such as BIOGLASS®, and mixtures thereof. Porous biocompatible ceramic materials currently commercially available include SURGIBONE® (CanMedica Corp., Canada), ENDOBON® (Merck Biomaterial France, France), CEROS® (Mathys, AG, Bettlach, Switzerland), and mineralized collagen bone grafting products such as HEALOS™ (DePuy, Inc., Raynham, Mass.) and VITOSS®, RHAKOSS™, and CORTOSS® (Orthovita, Malvern, Pa.). The framework can be a mixture, blend or composite of natural and/or synthetic materials.

In another embodiment, HSCs can be seeded onto, or contacted with, a felt, which can be, e.g., composed of a multifilament yarn made from a bioabsorbable material such as PGA, PLA, PCL copolymers or blends, or hyaluronic acid.

The HSCs can, in another embodiment, be seeded onto foam scaffolds that may be composite structures. Such foam scaffolds can be molded into a useful shape, such as that of a portion of a specific structure in the body to be repaired, replaced or augmented. In some embodiments, the framework is treated, e.g., with 0.1M acetic acid followed by incubation in polylysine, PBS, and/or collagen, prior to inoculation of the cells of the invention in order to enhance cell attachment. External surfaces of a matrix may be modified to improve the attachment or growth of cells and differentiation of tissue, such as by plasma-coating the matrix, or addition of one or more proteins (e.g., collagens, elastic fibers, reticular fibers), glycoproteins, glycosaminoglycans (e.g., heparin sulfate, chondroitin-4-sulfate, chondroitin-6-sulfate, dermatan sulfate, keratin sulfate, etc.), a cellular matrix, and/or other materials such as, but not limited to, gelatin, alginates, agar, agarose, and plant gums, and the like.

In some embodiments, the scaffold comprises, or is treated with, materials that render it non-thrombogenic. These treatments and materials may also promote and sustain endothelial growth, migration, and extracellular matrix deposition. Examples of these materials and treatments include but are not limited to natural materials such as basement membrane proteins such as laminin and Type IV collagen, synthetic materials such as EPTFE, and segmented polyurethaneurea silicones, such as PURSPAN™ (The Polymer Technology Group, Inc., Berkeley, Calif.). The scaffold can also comprise anti-thrombotic agents such as heparin; the scaffolds can also be treated to alter the surface charge (e.g., coating with plasma) prior to seeding with stem cells.

### 5.7 Preservation of HSCs

HSCs can be preserved, that is, placed under conditions that allow for long-term storage, or conditions that inhibit cell death by, e.g., apoptosis or necrosis. HSCs can be preserved using, e.g., a composition comprising an apoptosis inhibitor, necrosis inhibitor. In one embodiment, the invention provides a method of preserving a population of stem cells comprising contacting said population of stem cells with a stem cell collection composition comprising an inhibitor of apoptosis, wherein said inhibitor of apoptosis is present in an amount and for a time sufficient to reduce or prevent apoptosis in the population of stem cells, as compared to a population of stem cells not contacted with the inhibitor of apoptosis. In a specific embodiment, said inhibitor of apoptosis is a caspase-3 inhibitor. In another specific embodiment, said inhibitor of apoptosis is a JNK inhibitor. In a more specific embodiment, said JNK inhibitor does not modulate differentiation or proliferation of said stem cells. In another embodiment, said stem cell collection composition comprises said inhibitor of apoptosis and said oxygen-carrying perfluorocarbon in separate phases. In another embodiment, said stem cell collection composition comprises said inhibitor of apoptosis and said oxygen-carrying perfluorocarbon in an emulsion. In another embodiment, the stem cell collection composition additionally comprises an emulsifier, e.g., lecithin. In another embodiment, said apoptosis inhibitor and said perfluorocarbon are between about 0° C. and about 25° C. at the time of contacting the stem cells. In another more specific embodiment, said apoptosis inhibitor and said perfluorocarbon are between about 2° C. and 10° C., or between about 2° C. and about 5° C., at the time of contacting the stem cells. In another more specific embodiment, said contacting is performed during transport of said population of stem cells. In another more specific embodiment, said contacting is performed during freezing and thawing of said population of stem cells.

In another embodiment, the invention provides a method of preserving a population of HSCs comprising contacting said population of stem cells with an inhibitor of apoptosis and an organ-preserving compound, wherein said inhibitor of apoptosis is present in an amount and for a time sufficient to reduce or prevent apoptosis in the population of stem cells, as compared to a population of stem cells not contacted with the inhibitor of apoptosis.

Typically, during HSCs collection, enrichment and isolation, it is preferable to minimize or eliminate cell stress due to hypoxia and mechanical stress. In another embodiment of the method, therefore, a stem cell, or population of stem cells, is exposed to a hypoxic condition during collection, enrichment or isolation for less than six hours during said preservation, wherein a hypoxic condition is a concentration of oxygen that is less than normal blood oxygen concentration. In a more specific embodiment, said population of stem cells is exposed to said hypoxic condition for less than two hours during said preservation. In another more specific embodiment, said population of stem cells is exposed to said hypoxic condition for less than one hour, or less than thirty minutes, or is not exposed to a hypoxic condition, during collection, enrichment or isolation. In another specific embodiment, said population of stem cells is not exposed to shear stress during collection, enrichment or isolation.

### 5.8 Cryopreserved HSCs

The HSCS can be cryopreserved, e.g., in cryopreservation medium in small containers, e.g., ampoules. Suitable cryopreservation medium includes, but is not limited to, culture medium including, e.g., growth medium, or cell freezing medium, for example commercially available cell freezing medium, e.g., C2695, C2639 or C6039 (Sigma). Cryopreservation medium preferably comprises DMSO (dimethylsulfoxide), at a concentration of, e.g., about 5-10% (v/v). Cryopreservation medium may comprise additional agents, for example, methylcellulose and/or glycerol. HSCS are preferably cooled at about 1° C./min during cryopreservation. A preferred cryopreservation temperature is about −80° C. to about −180° C., preferably about −125° C. to about −140° C. Cryopreserved cells can be transferred to liquid nitrogen prior to thawing for use. In some embodiments, for example, once the ampoules have reached about −90° C., they are transferred to a liquid nitrogen storage area. Cryopreserved cells preferably are thawed at a temperature of about 25° C. to about 40° C., preferably to a temperature of about 37° C.

The HSCs disclosed herein can be preserved, for example, cryopreserved for later use. Methods for cryopreservation of cells, such as stem cells, are well known in the art. HSCs can be prepared in a form that is easily administrable to an individual. For example, provided herein are HSCs that are contained within a container that is suitable for medical use. Such a container can be, for example, a sterile plastic bag, flask, jar, or other container from which the HSCs can be easily dispensed. For example, the container can be a blood bag or other plastic, medically-acceptable bag suitable for the intravenous administration of a liquid to a recipient. The container is preferably one that allows for cryopreservation of the combined stem cell population. Cryopreserved HSCs can comprise HSCs derived from a single donor, or from multiple donors. The HSCs can be completely HLA-matched to an intended recipient, or partially or completely HLA-mismatched.

In another specific embodiment, the container is a bag, flask, or jar. In more specific embodiment, said bag is a sterile plastic bag. In a more specific embodiment, said bag is suitable for, allows or facilitates intravenous administration of the HSCs. The bag can comprise multiple lumens or compartments that are interconnected to allow mixing of the HSCs and one or more other solutions, e.g., a drug, prior to, or during, administration. In another specific embodiment, the composition comprises one or more compounds that facilitate cryopreservation of the combined stem cell population. In another specific embodiment, said HSCs is contained within a physiologically-acceptable aqueous solution. In a more specific embodiment, said physiologically-acceptable aqueous solution is a 0.9% NaCl solution. In another specific embodiment, said HSCs are HLA-matched to a recipient of said stem cell population. In another specific embodiment, said combined stem cell population comprises HSCs that are at least partially HLA-mismatched to a recipient of said stem cell population.

### 5.9 Pharmaceutical Preparations

As discussed above, one embodiment of the present disclosure is a pharmaceutical composition comprising a therapeutically effective amount of the human HSCs and a pharmaceutically acceptable carrier. The phrase “pharmaceutically acceptable” refers to molecular entities and compositions that are physiologically tolerable and do not typically produce an allergic or similar untoward reaction, such as gastric upset, dizziness and the like, when administered to a human, and approved by a regulatory agency of the Federal or a state government or listed in the U.S. Pharmacopeia or other generally recognized pharmacopeia for use in animals, and more particularly in humans. “Carrier” refers to a diluent, adjuvant, excipient, or vehicle with which the therapeutic is administered. Such pharmaceutical carriers can be sterile liquids, such as saline solutions in water and oils, including those of petroleum, animal, vegetable, or synthetic origin, such as peanut oil, soybean oil, mineral oil, sesame oil, and the like. A saline solution is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol, and the like. The composition, if desired, can also contain minor amounts of wetting or emulsifying agents, or pH buffering agents.

These compositions can take the form of solutions, suspensions, emulsions, tablets, pills, capsules, powders, sustained-release formulations, cachets, troches, lozenges, dispersions, suppositories, ointments, cataplasms (poultices), pastes, powders, dressings, creams, plasters, patches, aerosols, gels, liquid dosage forms suitable for parenteral administration to a patient, and sterile solids (e.g., crystalline or amorphous solids) that can be reconstituted to provide liquid dosage forms suitable for parenteral administration to a patient. Such compositions will contain a therapeutically effective amount of the compound, preferably in purified form, together with a suitable form of carrier so as to provide the form for proper administration to the patient. The formulation should suit the mode of administration.

Pharmaceutical compositions adapted for transdermal administration may be provided as discrete patches intended to remain in intimate contact with the epidermis of the recipient over a prolonged period of time.

Pharmaceutical compositions adapted for nasal and pulmonary administration may comprise solid carriers such as powders which can be administered by rapid inhalation through the nose. Compositions for nasal administration may comprise liquid carriers, such as sprays or drops. Alternatively, inhalation directly through into the lungs may be accomplished by inhalation deeply or installation through a mouthpiece. These compositions may comprise aqueous or oil solutions of the active ingredient. Compositions for inhalation may be supplied in specially adapted devices including, but not limited to, pressurized aerosols, nebulizers or insufflators, which can be constructed so as to provide predetermined dosages of the active ingredient.

Pharmaceutical compositions adapted for parenteral administration include aqueous and non-aqueous sterile injectable solutions or suspensions, which may contain anti-oxidants, buffers, bacteriostats, and solutes that render the compositions substantially isotonic with the blood of the subject. Other components which may be present in such compositions include water, alcohols, polyols, glycerine, and vegetable oils. Compositions adapted for parental administration may be presented in unit-dose or multi-dose containers, such as sealed ampules and vials, and may be stored in a freeze-dried (lyophilized) condition requiring only the addition of a sterile carrier, immediately prior to use. Extemporaneous injection solutions and suspensions may be prepared from sterile powders, granules, and tablets. Suitable vehicles that can be used to provide parenteral dosage forms of the invention are well known to those skilled in the art. Examples include: Water for Injection USP; aqueous vehicles such as Sodium Chloride Injection, Ringer's Injection, Dextrose Injection, Dextrose and Sodium Chloride Injection, and Lactated Ringer's Injection; water-miscible vehicles such as ethyl alcohol, polyethylene glycol, and polypropylene glycol; and non-aqueous vehicles such as corn oil, cottonseed oil, peanut oil, sesame oil, ethyl oleate, isopropyl myristate, and benzyl benzoate.

Selection of a therapeutically effective dose will be determined by the skilled artisan considering several factors which will be known to one of ordinary skill in the art. Such factors include the particular form of the inhibitor, and its pharmacokinetic parameters such as bioavailability, metabolism, and half-life, which will have been established during the usual development procedures typically employed in obtaining regulatory approval for a pharmaceutical compound. Further factors in considering the dose include the condition or disease to be treated or the benefit to be achieved in a normal individual, the body mass of the patient, the route of administration, whether the administration is acute or chronic, concomitant medications, and other factors well known to affect the efficacy of administered pharmaceutical agents. Thus, the precise dose should be decided according to the judgment of the person of skill in the art, and each patient's circumstances, and according to standard clinical techniques.

In certain embodiments, patients are treated with antipyretic and/or antihistamine (acetaminophen and diphenhydramine hydrochloride) to minimize any possible DMSO infusion toxicity related to the cryopreserve component in the HSCs treatment.

### 5.10 Assays

The transfected HSCs disclosed herein can be used in assays to determine the influence of culture conditions, environmental factors, molecules (e.g., biomolecules, small inorganic molecules. Etc.) and the like on stem cell proliferation, expansion, and/or differentiation, compared to HSCs not exposed to such conditions.

In a preferred embodiment, the HSCs are assayed for changes in proliferation, expansion or differentiation upon contact with a molecule. In one embodiment, for example, the invention provides a method of identifying a compound that modulates the proliferation of a plurality of HSCs, comprising contacting said plurality of HSCs with said compound under conditions that allow proliferation, wherein if said compound causes a detectable change in proliferation of said HSCs compared to a plurality of HSCs not contacted with said compound, said compound is identified as a compound that modulates proliferation of HSCs. In a specific embodiment, said compound is identified as an inhibitor of proliferation. In another specific embodiment, said compound is identified as an enhancer of proliferation.

In another embodiment, the invention provides a method of identifying a compound that modulates the expansion of a plurality of HSCs, comprising contacting said plurality of HSCs with said compound under conditions that allow expansion, wherein if said compound causes a detectable change in expansion of said plurality of HSCs compared to a plurality of HSCs not contacted with said compound, said compound is identified as a compound that modulates expansion of HSCs. In a specific embodiment, said compound is identified as an inhibitor of expansion. In another specific embodiment, said compound is identified as an enhancer of expansion.

In another embodiment, disclosed herein is a method of identifying a compound that modulates the differentiation of a HSCs, comprising contacting said HSCs with said compound under conditions that allow differentiation, wherein if said compound causes a detectable change in differentiation of said HSCs compared to a HSCs not contacted with said compound, said compound is identified as a compound that modulates proliferation of HSCs. In a specific embodiment, said compound is identified as an inhibitor of differentiation. In another specific embodiment, said compound is identified as an enhancer of differentiation.

In one embodiment, the HSC is obtained from a subject using the method in the present disclosure. In one embodiment, the subject is a patient with a specific disease or disorder. In one embodiment, the HSC is prepared from T-cells. In one embodiment, the iPS is prepared from fibroblasts.

### 5.11 HSC Gene Therapy of MLD: Clinical Testing

The present disclosure provides a method for clinically testing HSC Gene Therapy of MLD. Phase I and II clinical trial comprises following steps: 1. producing autologous HSC 2. 3^{rd} generation LV encoding ARSA, including self-inactivating LTR and internal PGK promoter. 3. busulfan-based conditioning. Detailed procedure of HSCGT is shown in FIG. 1.

### 6. EXAMPLES

The present invention may be better understood by reference to the following non-limiting examples, which are presented in order to more fully illustrate the preferred embodiments of the invention. They should in no way be construed to limit the broad scope of the invention.

### Example 6.1 Vector Designs for hARSA-LV

To manufacture a vector designed for LV-ARSA production, a lentiviral plasmid back bone AB.PCCL.sin.cPPT.u6miR-10-decoy.hpgk.gfp.wpre is purchased from Addgene (Addgene plasmid #46602; http://n2t.net/addgene:46602; RRID:Addgene_46602). u6-miR-10-Decoy was removed with BsmBI and the vector is re-ligated. GFP is then removed with AgeI and SalI. (IDF)

SalI-WPRE-SacII was subcloned by PCR into site directed mutagenesis (SDM) vector. WPRE is mutated by replacing ATG to AGG; SDM vector is now SalI-WPRE-mutated-SacII. hARSA cDNA is subcloned by PCR into SDM WPRE.muta1. SDM vector is then ageI-hARSA-salI-WPRE.mut1-sacII. GFP.WPRE cassette is subsequently replaced with hARSA.WPRE.mut1 cassette using AgeI and SaclI digest. (IDF)

The end transgene vector is propagated in 293FT cells with lentiviral plasmid MDL/VSV.G.

The schematic design of the plasmid construction is shown in FIG. 1.

### Example 6.2 Protocols for Mass Production of LV-ARSA for Clinical Application Fulfilled GMP Requirements

Optimal transfection conditions are designed based on the ratio of vectors LV-ARSA:MLD: to vesicular stomatitis G protein (VSV-G). LVs pseudo-typed with the VSV-G protein were generated by transfection into 293T cells of a packaging construct, a plasmid producing the VSV-G envelope, a plasmid containing the Rev-responsive element and the vector was prepared. For a high level of titter LV production, the ratio of core plasmids and package plasmids are 4:2:1:1, achieving high yields of LV. (Zanta-Boussif, 2009)

For mass LV production, serum free or low serum (3%) 293 FT culture medium free of antibiotics is used. For downstream processing of clinical grade LV production, a new recipe is developed to preserve LV at −80 degree without losing LV activity: 0.5-5% human albumin+1% heparin+0.9% NaCl. (IDF)

### Example 6.3 Protocols for Transduction of ARSA-LV into HSC

Cells were transduced with the LVs in according to the followed protocol. We use a recombinant human fibronectin fragment (RetroNectin) coated assay for LV attachment and LV suspension in medium to achieve high yield HSC infection. RetroNectin reagent dramatically enhances the efficiency of gene transduction into hematopoietic stem cells by retrovirus vectors (Hanenberg et al. 1996). After two hours of coating, the virus was removed from the culture flank, the human HSC was transferred into the LV virus-coated culture plate with the concentration of 5×10\hat{ }6/ml. Six hours late, the cells were collected for ARSA expression. The western blot and qPCR were used to evaluate the infection efficiency. The ARSA expression should be improved significantly upon the LV transduction.

### Example 6.4 In Vitro Medium to Maintain and Culture HSC with Stemness Multi-Pluripotency

To enhance HSC multipotency and sensitivity to LV infection, the HSC are cultured in a media comprising TPO, SCF, FLT3, IL-3 factors, and kinase inhibitors that inhibit mTOR and ROCK. The detailed receipt is: X-Vivo medium (sigma) 500 ml; TPO, 5-50 ng/ml; SCF, 5-50 ng/ml; Flt3, 5-50 ng/ml; IL-3, 10-50 ng/ml; Protamines, 2 ug/ml. See Table 1.

### Example 6.4.1 Cryopreservation Media for Stem Cells

The media for cryopreservation of stem cells comprise CP1, glucose, Albumin 2-20% (CP1 30%, glucose, 70 mg/L, 0.9% NaCl %. See Table 1.

TABLE 1
Media Ingredients

| | Ingredients | Full Name (if any) | Source, Product ID | Key Ingredient(s) |
|---|---|---|---|---|
| Example 6.4 | X-Vivo 15 | X-Vivo medium | Lonza Catalog | Yes |
| In vitro | medium | 500 ml | #: BE02-060F | |
| medium | 500 ml | X-VIVO ™ 15, Serum- | | |
| to maintain | | free hematopoietic | | |
| and culture | | cell medium, with L- | | |
| HSC with | | Glutamine, gentamicin and | | |
| stemness | | phenol red, xenofree, | | |
| multi pluripotency | | 500 mL | | |
| | TPO, 5-50 ng/ml | Thrombopoietin | Peprotech | Yes |
| | SCF, 5-50 ng/ml | Stem cell factor | Peprotech | Yes |
| | Flt3, 5-50 ng/ml | FMS-like tyrosine kinase-3 | Peprotech | Yes |
| | IL-3, 10-50 ng/ml | Interleukin 3 | Peprotech | Yes |
| | Protamines, | Protamines | Sigma | Yes |
| | 2 ug/ml | | | |
| Example | CP-1 ™ 30% | | Kyokuto | Yes |
| 6.4.1 | | | Pharmaceutical | |
| Cryopreservation | | | Industrial, | |
| media for | | | Tokyo Japan | |
| Stem cells | Glucose, | | | Yes |
| | 70 mg/L | | | |
| | Albumin 2-20% | | | Yes |

### Example 6.5 Transplantation Procedure

A batch of hematopoietic stem cells were collected for backup use. Then, another batch of patient's HSCs was collected and transduced with lentiviral-mediated functional genes of ARSA. The gene-corrected HSC was frozen and samples were examined to ensure they were functional and ARSA enzyme activity elevated, with no other contaminants. Finally, following a conditioning regimen, the gene-corrected cells were reinfused to the patient. The stem cells may be transplanted to the patients immediately following gene modification. Alternatively, the genetically modified stem cells may be frozen and performed a series of quality control checks before transplanting the cells to the patient. This allows more time to check the critical parameters of stem cells and gene function, thus reducing the risk of transplantation failure or severe complications. The patient's blood system was fully engrafted and a high level of ARSA enzyme was detected following transplantation. 40 months after transplantation, the patient's condition remains stable and satisfactory progress is made with rehabilitation.

### Example 6.6 Improvement of Mobility Post-Transplantation

From 2012 to 2014, patient had to spend 2 weeks per month in hospital due to her cognitive and motor deterioration. In 2014 September, patient successfully finished chemo-conditioning and transplantation. In 2015, the patient showed improved mobility and balance capacity post-transplantation.

A patient at the age of 13 years old, she has drowsiness and decline in memory, mobility, writing and speech. She was incorrectly diagnosed and treated for attention deficit disorder for more than 2 years. At age 15, she was finally diagnosed with MLD, confirmed by low ARSA activity with ARSA gene mutation, MRI imaging and clinical symptoms. Supportive treatment failed to arrest the progress of the disease. Prior to stem cell gene transplantation, she was unable to walk up and down the stairs, unable to manage personal hygiene and experienced episode seizures, severe constipation and occasional urinary incontinence. At 17, the hematopoietic stem cell gene transplantation was performed. Early results indicate stabilization of the disease. Her mobility and balance, capacity of hygiene management and ability to write have improved, constipation and urinary problems have resolved and no seizure have been recorded.

### Example 6.7 Vector Copy Number

Table 2 shows vector copy numbers.

| | | | | | |
|---|---|---|---|---|---|
| 0.210643 | 4465.431 | 34951.46 | 0.255522 | 0.235945 | |
| 0.040644 | 1031.952 | 41286.25 | 0.04999 | 0.030414 | |
| 0.18052 | 5618.765 | 51242.31 | 0.219302 | 0.199725 | |
| #VALUE! | 6103.265 | 38387.48 | 0.317982 | 0.298405 | |
| 0.059727 | 3504.389 | 41551.91 | 0.168675 | 0.149099 | |
| | | 0 | 3 | 6 | 12 |
| | CD3 | 0 | 0.040644 | 0.030414 | 0.087855 |
| | CD14 | 0 | 0.18052 | 0.199725 | 0.097083 |

### Example 6.8 Clinical Protocol and Patients

Symptomatic MLD patients are enrolled in the clinical trial from Shenzhen Second People's Hospital, the First Affiliated Hospital of Shenzhen University. Safety and efficacy of lentiviral hemoatopoietic stem cell gene therapy for MLD were evaluated.

<Inclusion Criteria>

For MLD inclusion criteria: 1. Confirmed diagnosis as MLD by ARSA genetic diagnosis; MRI imaging and low ARSA A activity (below 20% or normal level). The patient' symptoms and lesions have not been developed to the end stage of MLD.

<Exclusion Criteria>

For MILD exclusion criteria: 1. No clinical symptoms of MLD; 2. ARSA Activity >50% of healthy controls; 3. End stage of MLD; 4. Other complications, i.e. Cancer; 5. HIV RNA and/or HCV RNA and/or HBV DNA positive patients; 6. Patient who underwent allogenic hematopoietic stem cell transplantation with evidence of residual cells of donor origin. Sample size for both intervention groups are 16. For the first intervention group, the patients were administered with lentiviral hematopoietic stem cell gene therapy. For the control group, no gene therapy was administered. Neural scores and ARSA activity (MLD) are measured. Blood samples were collected from patients.

1. A recombinant lentiviral vector (LV) comprising: an arylsulfatase A (ARSA) gene encoding an arylsulfatase A polypeptide, where said LV is a self-inactivating (SIN) lentiviral vector.

2. The vector of claim 1, wherein the vector comprises a recombinant ARSA gene under the control of an ARSA gene 5′ promoter and an ARSA 3′ enhancer.

3. The vector of claim 1, wherein the vector comprises an insulator.

4. The vector of claim 1, wherein the vector comprises a Rev Responsive Element (RRE).

5. The vector of claim 1, wherein the vector comprises a central polypurine tract.

6. The vector of claim 1, wherein the vector comprises a post-translational regulatory element.

7. The vector of claim 6, wherein the posttranscriptional regulatory element is a modified Woodchuck Post-transcriptional Regulatory Element (WPRE).

8. The vector of claim 7, wherein the modified WPRE has a nucleic acid sequence as set forth in SEQ ID NO: 3.

9. The vector of claim 1, wherein the ARSA gene is human ARSA gene.

10. The vector of claim 9, wherein the ARSA gene has a nucleic acid sequence as set forth in SEQ ID NO: 2.

11. A host cell transduced with the vector of claim 1.

12. The host cell of claim 11, wherein the cell is a stem cell.

13. The host cell of claim 12, wherein said cell is a stem cell derived from bone marrow.

14. The host cell of claim 11, wherein the cell is a hematopoietic stem cell.

15. A method of maintaining the multipotency and increased sensitivity for LV infection of the host cell of claim 11, comprising incubating the host cells in a media comprising TPO, SCF, FLT3, and IL-3 factors.

16. The method of claim 15 wherein the media further comprises kinase inhibitors that inhibit mTOR and ROCK activity.

17. The method of claim 15 wherein the media comprises CP1, glucose, albumin at 2-20% and Herceptin.

18. The method of claim 17 wherein the albumin is at 5-10%.

19. The medium of claim 17, wherein the medium is frozen.

20. A method of treating Metachromatic leukodystrophy (MLD) in a subject, said method comprising: transducing a stem cell from said subject with a vector of claim 1; and transplanting said transduced cell into said subject where said cells express said ARSA gene.

21. The method of claim 20 wherein the cell is a stem cell derived from bone marrow.

22. The method of claim 20 wherein the cell is a human hematopoietic stem or progenitor cell.

23. The method of claim 22, wherein the method increases the chances of survival of the hematopoietic stem or progenitor cell during gene therapy.

24. The method of claim 22, wherein the method prevents apoptosis of the hematopoietic stem or progenitor cell.

25. A method of regulating expression of a transgene comprising delivering the vector of claim 1 to a hematopoietic stem or progenitor cell.

26. A pharmaceutical composition comprising the vector of claim 1 for treating Metachromatic leukodystrophy (MLD) in a subject.
